(19) United States
(12) Patent Application Publication
(10) Pub. No.: US 2022/0409602 A1
(43) Pub. Date: Dec. 29, 2022

(54) DOPAMINE D3 RECEPTOR LIGANDS FOR TREATMENT OF DYSKINESIA

(71) Applicant: TEXAS BIOMEDICAL RESEARCH INSTITUTE, San Antonio, TX (US)

(72) Inventor: Marcel M. Daadi, San Antonio, TX (US)

(73) Assignee: TEXAS BIOMEDICAL RESEARCH INSTITUTE, San Antonio, TX (US)

(21) Appl. No.: 17/840,563

(22) Filed: Jun. 14, 2022

Related U.S. Application Data
(60) Provisional application No. 63/202,489, filed on Jun. 14, 2021.

(51) Int. Cl. A61K 31/4535; A61P 25/14

(52) U.S. Cl. CPC A61K 31/4535; A61P 25/14

(57) ABSTRACT

Provided here are arylpiperazine pharmacophore compositions that mitigate levodopa-induced dyskinesia and significantly improve Parkinson's disease-like symptoms. These dopamine D3 receptor ligands have high affinity and selectivity, are orally active, and have desirable drug-like properties.

### CROSS-REFERENCE TO RELATED APPLICATIONS

This application claims the benefit of and priority to U.S. Provisional Application No. 63/202,489, filed Jun. 14, 2021, which is incorporated by reference herein in its entirety.

### GOVERNMENT SUPPORT

This invention was made with government support under grant numbers: R56 AG059284 and OD P51 OD011133 awarded by National Institutes of Health. The government has certain rights in the invention.

### TECHNICAL FIELD

The disclosure relates to dopamine D3 receptor ligand compositions and methods of use of these compositions for treatment of dyskinesia. These compositions are also used specifically in levodopa-induced dyskinesia experienced in Parkinson's disease (PD).

### BACKGROUND

PD is a complex multisystem, chronic and so far incurable disease with significant unmet medical needs. In the United States, over a million people suffer from PD and 60,000 new cases are diagnosed each year with an estimated cost of USD 27 billion per year. As the incidence of PD increases with aging, with an additional trend of increased incidence in men over the age of 70, the expected burden will continue to escalate with the aging population. PD pathology is associated primarily with the death of dopamine neurons in the substantia nigra and manifests with motor and non-motor dysfunctions including tremor, bradykinesia, rigidity, cognitive deficits, and sleep disturbances.

These symptoms are managed by dopamine-replacement pharmacological treatments aiming at enhancing dopamine in the striatum with the dopamine precursor L-3,4-dihydroxy-phenylalanine (levodopa). Since its discovery in the 1961, levodopa remains the gold standard pharmacotherapy as it produces effective relief of the motor symptoms. However, the progressive nature of PD associated with the degenerative process within and beyond the nigrostriatal system causes a multitude of side effects within 5 years including the uncontrolled involuntary movements or levodopa-induced dyskinesia, the “wearing OFF” and the “ON/OFF” motor fluctuations. Over 80% of Parkinsonian patients treated with levodopa develop levodopa-induced dyskinesia after five years. These debilitating side effects spurred the discovery of alternative dopamine-replacement pharmacological treatments. Examples are general dopamine receptor agonists, inhibitors of enzymes that degrade dopamine, and the anti-dyskinetic drug in current use, amantadine. These alternative treatments helped mitigate some of the side effects. Unfortunately, each drug elicits a new range of additional side effects making it challenging to manage the disease. Surgical treatments including deep brain stimulation (DBS) are alternatives that improve PD symptoms and dyskinesia. However, DBS has potential adverse events associated with the surgical procedure, the device, and stimulation and its loss of benefit parallels the progressive degenerative changes associated with PD.

### SUMMARY

Provided here are compositions and methods to address these shortcomings of the art and provide other additional or alternative advantages. Embodiments include methods of treating dyskinesia by administering an effective amount of a dopamine receptor 3 ligand containing an arylpiperazine pharmacophore or a pharmaceutically acceptable salt, derivative, hydrate or solvate thereof. In certain embodiments, the dyskinesia is levodopa-induced dyskinesia. In certain embodiments, the dopamine receptor 3 ligand is a N-(4-(4-phenyl piperazin-1-yl)butyl)-4-(thiophen-3-yl)benzamide D3 ligand. In certain embodiments, the dopamine receptor 3 ligand is PD13R (as shown in Formula 1 below). In certain embodiment, the dopamine receptor 3 ligand is administered as a thiophenyl benzoate salt of PD13R.

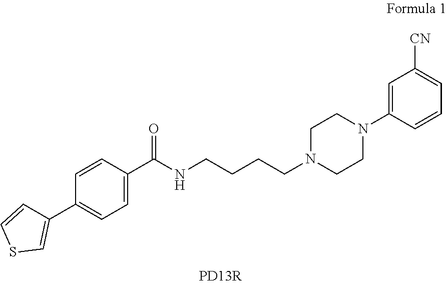 [SMILES: N#Cc1cccc(N2CCN(CCCCNC(=O)c3ccc(-c4ccsc4)cc3)CC2)c1]

In certain embodiments, the dopamine receptor 3 ligand containing an arylpiperazine pharmacophore or a pharmaceutically acceptable salt, derivative, hydrate or solvate thereof, also contains a phenyl thiophene moiety. In certain embodiments, the arylpiperazine pharmacophore and the phenyl thiophene moiety are separated by a spacer. In certain embodiments, the dopamine receptor 3 ligand containing an arylpiperazine pharmacophore or a pharmaceutically acceptable salt, derivative, hydrate or solvate thereof, also contains one or more of a heterocyclic ring, such as triazole, imidazole, pyrimidine, pyrrazole, and pyridine. In certain embodiments, the dopamine receptor 3 ligand contains one or more of a piperazine bioisostere and a bicyclic bioisostere, or a pharmaceutically acceptable salt, derivative, hydrate or solvate thereof. In certain embodiments, the dopamine receptor 3 ligand is orally administered at a dosage ranging from about 1 mg/kg to about 20 mg/kg.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will be readily understood by the following detailed description in conjunction with the accompanying drawings. These drawings illustrate the principles of the disclosure and no limitation of the scope of the disclosure is thereby intended.

FIGS. 1A-1D are images from the docking simulation analysis for PD13R and SWR-3-73 in D3R and D2R, respectively. FIG. 1A illustrates molecular recognition of PD13R (blue) in D3R cryo-EM structure (grey) showing interactions between the cyano (CN) group of the PD13R and residues TYR 365 and ASP 110 at the orthosteric binding site. There were polar contacts between the PD13R and the D3R including with the TYR 365 and ASP 110 residues. FIG. 1B illustrates a representative pose of PD13R, showing short contact with TYR 365 and longer-range cyano group contact to SER 182 in flexible loop at pocket opening. FIG. 1C illustrates a pose of compound SWR-3-73 interacting by polar contact at residue SER 182. FIG. 1D illustrates structural superposition of PD13R-bound D2R (blue) demonstrating the difference in cavity filling between the 2 protein receptors D3R and D2R. D2R residues TYR 403 and ILE 283, representing S1831 point mutation in D2R. Two potential points of polar contact with the ligands are lost.

FIGS. 2A and 2B are graphical representations demonstrating that Parkinsonian marmosets develop levodopa-induced dyskinesia. To develop dyskinesia, marmosets were treated with increasing dose of levodopa/Carbidopa (1:1) starting with doses of 5, 10 and 15 mg/kg followed by a maximum dose of 20 mg/kg, which was maintained for 6 weeks. In FIG. 2A, dyskinesia was measured weekly from week 1 of treatment to week 6 and analyzed as described hereinafter. FIG. 2A is a graphical representation of the dyskinesia score over time. FIG. 2B is the quantitative analysis of the abnormal activity of the animals registered by the actiwatch-mini during the period of dyskinesia induced in Parkinsonian marmosets previously exposed to levodopa and acutely challenged with high dose 20 mg/kg of levodopa, compared to vehicle treated and 2 months (M) wash-off period. Arrow indicates time of the administration of vehicle or levodopa to the animals. Levodopa treatment resulted in a marked increase in motor activity of the Parkinsonian marmosets reaching the ON-period and crosses the dyskinesia threshold (dotted line) within 30 min after injection. At this stage, the peak-dose dyskinesia and the ON response coincide. The animals return to the ON-period without dyskinesia approximately 145 minutes after Levodopa treatment and gradually enter the wearing-OFF period. Statistical analysis was performed using two-way ANOVA followed by Bonferroni post-hoc test analysis. ***P<0.001. Error bars represent standard error.

FIGS. 3A-3C are graphical representations demonstrating that treatment with the dopamine D3R ligand PD13R prevents the expression of levodopa-induced dyskinesia in the Parkinsonian marmosets. FIG. 3A is a graphical representation of the dose-range analysis of PD13R anti-dyskinetic effects. Dyskinesia was induced in Parkinsonian marmosets previously exposed to levodopa by acute challenge with 20 mg/kg of levodopa combined with increasing doses of PD13R compound. The anti-dyskinetic effects of PD13R were dose-dependent with optimal outcome at 10 mg/kg. FIG. 3B is a graphical representation of the elimination of the levodopa-induced dyskinesia at the optimal dose of PD13R (10 mg/kg) and improvement of the Parkinson's Disease Rating Scale (PDRS) in dyskinetic Parkinsonian marmosets. FIG. 3B is a graphical representation of the quantitative analysis of the motor activity of the animals recorded by the actiwatch-mini during levodopa-induced dyskinesias in marmosets treated with 20 mg/kg of levodopa in the presence of increasing doses of PD13R. Arrow indicates time of drug administration. Levodopa treatment resulted in a marked hyperactivity that surpassed the therapeutic ON-period and crossed the dyskinesia threshold (dotted line) during the first 50 min after drug administration for all doses except the therapeutic dose 10 mg/kg. At the later dose, motor activity remained within the therapeutic ON-period zone without dyskinesia. Statistical analysis was performed using two-way ANOVA followed by Bonferroni post-hoc test analysis. *P<0.05, **P<0.01, ***P<0.001, ns: Not Significant. Error bars represent standard error.

FIGS. 4A-4B are graphical representations demonstrating that anti-dyskinetic effects of PD13R compared to a D3R partial agonist SWR373 and to the NMDA glutamate receptor inhibitor, amantadine. FIG. 4A is a graphical representation of the dyskinesia that was induced in Parkinsonian marmosets previously exposed to levodopa by acute challenge with 20 mg/kg of levodopa combined with therapeutic doses (10 mg/kg) of: (1) PD13R with arylpiperazine-based pharmacophore versus (2) SWR-3-73 with diazaspiro-based pharmacophore and (3) amantadine. FIG. 4B is a graphical representation of the quantitative analysis of the time course motor activity of the animals recorded by the actiwatch-mini during levodopa-induced dyskinesias in marmosets treated with 20 mg/kg of levodopa in the presence of PD13R, SWR-3-73, and amantadine. Arrow indicates time of drug administration. At the therapeutic dose of 10 mg/kg dose, PD13R and amantadine inhibited the expression of dyskinesia compared to SWR-3-73 treatment. Both PD13R and Amantadine maintained the motor activity within the ON-period without dyskinesia. Statistical analysis was performed using two-way ANOVA followed by Bonferroni post-hoc test analysis. ***P<0.001. Error bars represent standard error.

FIGS. 5A-5J are representative actograms of Parkinsonian marmosets throughout 24-hour daytime and nighttime period (9 am to 9 am next day) recording animal activities, and representative actograms of the nocturnal activity (7 pm to 6:30 am). All actograms were recorded during the OFF period, and during levodopa-induced dyskinesias in marmosets acutely challenged with 20 mg/kg of levodopa in the presence of vehicle, PD13R, SWR-3-73, or amantadine. Amantadine causes sleep disturbances in dyskinetic Parkinsonian marmosets under levodopa treatment. The data shows a clear abnormal increase in nocturnal activity in dyskinetic animals treated with amantadine while both PD13R and SWR-3-73 treated animals exhibited reduced baseline-level nocturnal activity.

FIGS. 6A-6D are graphical representations demonstrating amantadine worsens sleep quality in Parkinsonian marmosets. FIGS. 6A and 6C are graphical representations of the quantitative analysis of sleep quality parameters showed that dyskinetic marmosets exhibited a significant increase in nocturnal activity under amantadine treatment, while neither PD13R under increasing doses (A) nor SWR-3-73 at 10 mg/kg (C) modified the nocturnal activity of the animals. FIGS. 6B and 6D are graphical representations of the sleep efficiency that also significantly dropped under amantadine treatment compared to PD13R administered at increasing doses (FIG. 6B) and to SWR-3-73 at 10 mg/kg (FIG. 6D). Statistical analysis was performed using two-way ANOVA followed by Bonferroni post-hoc test analysis. *P<0.05, **P<0.01, ***P<0.001. Error bars represent standard error.

### DETAILED DESCRIPTION

Dopamine actions are mediated through five-member family of G-protein-coupled receptors (GPCRs) classified into two subtypes, D1-like and D2-like receptors based on sequence homology, G-protein coupling and pharmacology. D1R and D5R are coupled to the stimulatory D1-like G-protein family members (G_{s/olf}), while D2R, D3R, and D4R are coupled to the inhibitory D2-like G-protein family members (G_{i/o}). Although dopamine D3 receptor (D3R) has been implicated in PD and levodopa-induced dyskinesia, the high sequence homology between the D3R and D2R, including in the transmembrane region and the orthosteric binding site that binds dopamine, has made it challenging to translate promising leads to the clinic.

D3R plays critical role in the central nervous system health and disease. Although it is well known for its involvement in drug abuse and addiction, reward, cognition, schizophrenia, impulse control disorders and Parkinson's disease, D3R has been implicated in a variety of other functions including neuroinflammation, neurogenesis, protein aggregation and neurotrophic factor secretion. The expression of D3R in Parkinson's disease animal models and patients mirrors the dynamic state of the disease impacted by the timing of the PD-like symptoms development, the evolution of the neurodegenerative process and the therapeutic intervention such as the levodopa treatment. Previous reports have also suggested that D3R expression is species specific, in rat models the D3R expression is limited to dopaminergic areas known to be associated with cognitive and emotional functions. In the NHP models, the D3R expression closely resemble human pattern with a widespread expression in motor and associative structures of the basal ganglia and in limbic system. In all these species however, D3R is down regulated in the basal ganglia after the stabilization of PD-like symptoms and D3R increases in expression and binding in caudate, putamen and globus pallidus after levodopa treatment and development of levodopa-induced dyskinesia. D3R was investigated for its role in the genesis and expression of levodopa-induced dyskinesia and as a viable therapeutic target for treatment of such effects.

In both rodent and nonhuman primate models of PD with levodopa-induced dyskinesia, D3R expression is decreased during the development of PD-like symptoms and is abnormally up-regulated in the caudate and putamen after prolonged levodopa treatment and levodopa-induced dyskinesia expression. These data suggest the involvement of D3R in the pathogenesis of motor complications and levodopa-induced dyskinesia following levodopa pharmacotherapy.

Two novel D3 receptor ligands were investigated, PD13R and SWR-3-73, which belong to two separate classes of pharmacophores: the arylpiperazine and the diazaspiro, respectively in treating levodopa-induced dyskinesia in a nonhuman primate model of PD. Both compounds had high affinity and selectivity for D3R and they acted as partial agonists via G-protein by inhibiting the adenyl cyclase signaling pathway and decreasing cAMP production. PD13R inhibited the expression of levodopa-induced dyskinesia while improving motor functions and sleep efficiency in dyskinetic animal models. SWR-3-73 did not inhibit expression of levodopa-induced dyskinesia nor the Parkinson's-like symptoms.

Embodiments include methods of treating dyskinesia by administering an effective amount of a dopamine receptor 3 ligand containing an arylpiperazine pharmacophore or a pharmaceutically acceptable salt, derivative, hydrate or solvate thereof. In certain embodiments, the dyskinesia is levodopa-induced dyskinesia. In certain embodiments, the dopamine receptor 3 ligand is a N-(4-(4-phenyl piperazin-1-yl)butyl)-4-(thiophen-3-yl)benzamide D3 ligand. In certain embodiments, the dopamine receptor 3 ligand is PD13R. In certain embodiment, the dopamine receptor 3 ligand is administered as a thiophenyl benzoate salt of PD13R.

In certain embodiments, the dopamine receptor 3 ligand containing an arylpiperazine pharmacophore or a pharmaceutically acceptable salt, derivative, hydrate or solvate thereof, also contains a phenyl thiophene moiety. In certain embodiments, the arylpiperazine pharmacophore and the phenyl thiophene moiety are separated by a spacer. In certain embodiments, the dopamine receptor 3 ligand containing an arylpiperazine pharmacophore or a pharmaceutically acceptable salt, derivative, hydrate or solvate thereof, also contains one or more of a heterocyclic ring, such as triazole, imidazole, pyrimidine, pyrrazole, and pyridine. In certain embodiments, the dopamine receptor 3 ligand contains one or more of a piperazine bioisostere and a bicyclic bioisostere, or a pharmaceutically acceptable salt, derivative, hydrate or solvate thereof. In certain embodiments, the dopamine receptor 3 ligand is orally administered at a dosage ranging from about 1 mg/kg to about 20 mg/kg. In certain embodiments, the dopamine receptor 3 ligand is administered at a dosage ranging from about 5 mg/kg to about 20 mg/kg. In certain embodiments, the dopamine receptor 3 ligand is administered at a dosage ranging from about 5 mg/kg to about 15 mg/kg.

Embodiments include methods of treating levodopa-induced dyskinesia in a subject in need thereof by administering to the subject an effective amount of a dopamine receptor 3 ligand containing N-(4-(4-phenyl piperazin-1-yl)butyl)-4-(thiophen-3-yl)benzamide or a pharmaceutically acceptable salt, derivative, hydrate or solvate thereof. In certain embodiments, the dopamine receptor 3 ligand containing N-(4-(4-phenyl piperazin-1-yl)butyl)-4-(thiophen-3-yl)benzamide or a pharmaceutically acceptable salt, derivative, hydrate or solvate thereof is administered orally. In certain embodiments, the dopamine receptor 3 ligand containing N-(4-(4-phenyl piperazin-1-yl)butyl)-4-(thiophen-3-yl)benzamide or a pharmaceutically acceptable salt, derivative, hydrate or solvate thereof is administered as a thiophenyl benzoate salt. In certain embodiments, the dopamine receptor 3 ligand is administered at a dosage ranging from about 1 mg/kg to about 20 mg/kg. In certain embodiments, the dopamine receptor 3 ligand is administered at a dosage ranging from about 5 mg/kg to about 20 mg/kg. In certain embodiments, the dopamine receptor 3 ligand is administered at a dosage ranging from about 5 mg/kg to about 15 mg/kg.

Embodiments include pharmaceutical compositions containing an effective amount of the D3 receptor partial agonist, PD13R (arylpiperazine-based pharmacophore). PD13R significantly suppresses the expression of levodopa-induced dyskinesia in a nonhuman primate model of PD. SWR-3-73 (diazaspiro-based pharmacophore) did not inhibit expression of levodopa-induced dyskinesia. The anti-dyskinetic effects of PD13R didn't affect the anti-Parkinsonian benefits of levodopa while improving sleep efficiency compared to amantadine. In certain animal models, PD13R demonstrated desirable CNS drug-like features with high affinity for the D3R (Ki=0.50 nM), high selectivity for the D3R over the D2R (1486-fold), >20,000-fold selectivity for D3R over D1R or D5R and >1600-fold selectivity for D3R over D4R. PD13R exhibited low efficacy in the forskolin-dependent adenylyl cyclase inhibition assay (19.4% maximum activity) and a prolonged half-life (>60 m in human and rat liver microsome assays) of over 10 hrs in the in vivo PK study. Together, these data demonstrated that PD13R is efficacious in treating levodopa-induced dyskinesias, and is orally active with desirable drug-like properties, including potency, selectivity, and bioavailability.

Levodopa-induced dyskinesia develops in up 95% of patients after 15 years of treatment. Yet, it remains the gold standard pharmacotherapy for PD. Attenuating dyskinesia has been a significant challenge in the clinical management of Parkinson's disease. The attempt to attenuate dyskinesia by reducing or adjusting the dose of levodopa and implementing other adjunctive dopamine-replacement drugs produced inconsistent outcome. PD13R eliminates the expression of levodopa-induced dyskinesia without affecting the anti-Parkinsonian effects of Levodopa. This lead compound PD13R is a D3R partial agonist demonstrated in the forskolin-dependent adenylyl cyclase inhibition assay producing 19.4% of the activity of the maximum response observed with quinpirole.

The anti-dyskinetic effects of PD13R were similar to those observed with amantadine treatment, the currently prescribed drug for dyskinesia that was used as positive control. Based on several clinical studies, amantadine, which is an NMDA glutamate receptor inhibitor, was designated as efficacious for the treatment of dyskinesia by the Movement Disorder Society Evidence Based Medical Review. Interestingly, different effects on sleep patterns were detected between amantadine and the two D3R partial agonists. Both PD13R and SWR-3-73 did not affect sleep efficiency, while animals treated with amantadine exhibited a significant increase in nocturnal activity and decrease in sleep efficiency. Previous reports have highlighted the potential side effects of amantadine with the most common including cardiovascular dysfunction, myoclonus, orthostatic hypotension, peripheral edema, urinary tract infection, nervousness, insomnia, anxiety, hallucinations, delirium, confusion, nausea and constipation. Thus, better anti-dyskinetic agents remain a critical medical need. This novel D3R partial agonist, PD13R has demonstrated a high sub-nanomolar affinity for the D3R (Ki=0.50 nM), 1486-fold higher selectivity for the D3R over the D2R, 20,000-fold higher selectivity for D3R over D1R or D5R and over 1600-fold selectivity for D3R over D4R. PD13R has a prolonged half-life of over 10 hours in vivo, and a partial agonistic activity (19.4% maximum activity) in the forskolin-dependent adenylyl cyclase inhibition assay.

In the CNS disorders, improving the D3 versus D2 compound selectivity has been the most challenging aspect in developing selective D3R ligands. The sequence similarity between D3R and D2R is 90%, while sequence identities in relevant interactions sites with ligands such as the transmembrane region are highest for D2 versus D3 at 79%. The docking simulation analysis for PD13R and SWR-3-73 reveals high selectivity for D3R versus D2R. The overlay of D3R and D2R crystal structures docking pose of the PD13R revealed a loss of the polar contact at the key residues TYR 365 and SER 182, the TYR 365 became TYR 408 and reoriented completely away from the pocket leading to loss of the polar contact with the ligand. Furthermore, the bottom part of the binding pocket is unoccupied in the D2R. This un-filled portion of the pocket is important because cavity-filling connects selectivity to affinity, and the unfilled void would decrease selectivity and binding affinity to D2R. The optimal cavity filling of PD13R for D3R vs D2R could be mediated by the spacer linking the arylpiperazine to the arylamid, enabling a full cavity filling and improved selectivity and affinity to D3R. These finding are consistent with previous SAR studies demonstrated that the length of the spacer impact the affinity and selectivity to the D3R. Decreasing the spacer length from butylene to propylene or ethylene markedly reduces affinity to D3R; similar results were obtained when the spacer is increased from butylene to pentamethylene. The evidence show that even though both compounds PD13R and SWR-3-73 acted as partial D3R agonists, only the arylpiperazine-based scaffold was efficacious in treating dyskinesias. Substitution in the 4-position of the phenyl ring of the arylpiperazine lead to losses in binding to D2R and a 1486-fold high selectivity for D3R over D2R. Taken together, these data suggest that arylpiperazine, along with the spacer and the phenyl thiophene, drives the optimal activity and cavity filling of D3R. These findings are consistent with the structure activity relationships for D3R antagonists in substance abuse concluding that in order to obtain high affinity for D3R (<10 nm), high selectivity over D2R (>100 fold) the arylpiperazine-based scaffold are the most promising. Specifically, the structural features for potent and selective D3 antagonists include 1) an extended aryl amide, 2) piperazine containing an aromatic or heteroaromatic moiety in position 4 and 3) 4-atom (n-butyl) linking chain between the two functional groups. Although the arylpiperazine element is the primary pharmacophore, the selectivity for D3R also depends on the length of the linker. The arylpiperazine pharmacophore was mostly described as antagonist, which has created confusion in the Parkinson's disease field, as it is also a common structure of partial D3R agonist.

The lead compound PD13R, a partial D3R agonist, exerts approximately 19% adenylyl cyclase inhibition of a full agonist. Importantly, PD13R did not affect the therapeutic benefits of levodopa, manifested by the improvement in the Parkinson's disease rating score. The PD-like symptoms evaluated included tremors, bradykinesia, and posture and general activity measured by an accelerometer device, the Actiwatch mini. In addition, motor and cognitive functions were measured using the object retrieval task with barrier detour. A clear improvement in all PD-like symptoms and movement coordination were observed in animals treated with PD13R compared to control or those treated SWR-3-73.

As used herein, the terms “treating” or “treatment” of a subject includes the administration of an arylpiperazine pharmacophore-containing composition to a subject with the purpose of preventing, curing, healing, alleviating, relieving, altering, remedying, ameliorating, improving, stabilizing or affecting dyskinesia, or a symptom of Parkinson's disease. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By the term “effective amount” of a therapeutic agent is meant a nontoxic but sufficient amount of such agent to provide the desired effect. The amount of a therapeutic agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts. An “effective amount” of a drug necessary to achieve a therapeutic effect may vary according to factors such as the age, sex, and weight of the subject. Dosage regimens can be adjusted to provide the optimum therapeutic response. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments, a dosage of between 1 and 50 mg/kg may be appropriate. In other embodiments, a dosage of between 1 and 20 mg/kg may be appropriate. For example, several divided doses may be administered daily or the dose may be proportionally reduced as indicated by the exigencies of the therapeutic situation. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Embodiments include methods of treating dyskinesia by an oral administration of an effective amount of a dopamine receptor 3 ligand containing an arylpiperazine pharmacophore or a pharmaceutically acceptable salt, derivative, hydrate or solvate thereof. The terms “administered orally” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration.

### Examples

All the animals (n=3) have been previously treated with MPTP as described in detail. Briefly, MPTP (Sigma Aldrich) (dissolved in physiological saline) at a concentration of 2 mg/ml was subcutaneously injected (2 mg/kg B.wt) for 5 consecutive days. After a wash out period of 72 hours from last MPTP injection, the marmosets were returned to their home cages and monitored twice daily for rest of the study period.

For induction of dyskinesias, Levodopa (Sigma Aldrich) and carbidopa (Sigma Aldrich) at 1:1 were administered orally once daily for 5 days of a week. The drugs were mixed in either Ensure® pudding, cottage cheese, or marshmallows. The marmosets were started on lower dose (5 mg/kg B.wt) during the first week and gradually increased to 10 and 15 mg/kg B.wt over the course of next 2 weeks. Once the animals were acclimated to the drugs, the dose was increased to 20 mg/kg B.wt and continued for next 6 weeks. To test the anti-dyskinetic effects of the drugs, amantadine (Sigma Aldrich; 10 mg/kg B.wt) or the compounds PD13R and SWR-3-73 (both at 10 mg/kg B.wt) synthesized as previously described were orally administered along with Levodopa and carbidopa (1:1, 20 mg/kg B.wt) and the changes in dyskinesia symptoms and diurnal and nocturnal activities were analyzed using the dyskinesia disability score and the Actiwatch mini, respectively.

### Parkinson's Disease Rating Scale (PDRS)

The severity of symptoms in the marmosets was categorized using a validated Parkinsonian rating scale for NHP. The PDRS correlates highly with striatal dopamine concentrations detected by postmortem immunohistopathology and it is modeled on the Unified Parkinson's Disease Rating Scale (UPDRS) and the Hoehn & Yahr scale used clinically to categorize PD patients. The PDRS was performed in daylight by video recording animals for 30 minutes. The evaluation was carried out biweekly before and after MPTP injections. The videos were scored by a blinded operator using PDRS, with a maximal disability score of 57 in the following manner: 0=normal, 1=mild, 2=moderate, 3=severe; rest tremor, action tremor, tremor of the head, tremor right arm, tremor left arm, freezing, locomotion, fine motor skills right hand, fine motor skills left hand, bradykinesia right arm, bradykinesia left arm, posture, hypokinesia, balance, posture, startle response, gross motor skills right hand and gross motor skills left hand, apathy (defined as a state of indifference), vocalization, drooling or frothing, tongue/face/lips. Independently of the PDRS, rigidity was assessed by evaluating the resistance to passive joint movements and the range of motion during reaching for food. Prior to MPTP lesion, we trained animals using their favorite food (i.e. marshmallows) as reward. The marmosets were trained and conditioned to perform the rewarding visually guided task of reaching and grabbing a marshmallow. The evaluation was performed before and after MPTP.

### Dyskinesia Rating Scale

The severity of dyskinesia in the marmosets was categorized using a validated dyskinesia rating scale for NHPs. Dyskinesia was analyzed from 5-minute videos of the monkeys recorded at every 30 minutes on Tuesday, Wednesday and Thursday of each week. The videos were recorded between 9:30 AM-4:30 PM. Each day the vehicle or drugs were administered at 11:30 AM. The severity of dyskinesias was scored for different segments of the animal's body on a scale from 0 to 3, with 0=absent, 1=mild, 2=moderate, 3=severe. The body segments scored were: 1. Right upper limb dyskinesias (0-3); 2. Left upper limb dyskinesias (0-3); 3. Right lower limb dyskinesias (0-3); 4. left lower limb dyskinesias (0-3); 5. Trunk dyskinesias (0-3); 6, Head/facial dyskinesias (0-3). The severity of the rating was based on the frequency and amplitude of the abnormal movements. The total score is deducted from the six sub-scores to yield a dyskinesia score of 0 to 3: with 0=absent, 1=mild, manifesting by transient abnormal involuntary movements with choreic form, flicking of arms and limbs, increased running and hopping in the cages. 2=moderate, characterized by frequent (over 50%) and intermittent uncontrolled irregular movements of limbs interfering with normal activity, while the animals are able to reach for treats. 3=severe, characterized by a sustained dyskinesia manifesting with chorea and athetosis with sinuous writhing movements of the limbs, and dystonia with sustained extension of hind limbs and knees. The animals are unable to reach and grasp for treats (Video 1).

Object Retrieval Task with Barrier Detour (ORTBD)

The object retrieval task with a barrier detour is a reward based behavioral testing system that was previously described to evaluate motor and cognitive functions of NHP. Briefly, the task requires the test subject to retrieve a reward (marshmallow) fastened to a tray from the open side (bypassing the barrier) of a transparent box. For the current study, the testing apparatus was modified to fit the marmoset's home cage and the animals were acclimatized to the apparatus prior to testing. Behavioral analysis was done for 3 consecutive days with 20 trials per day before and 1 hr after administration of the drugs. All parameters measured were previously described in detail. During each trial the orientation of the open side of the box was randomly changed to either left or right of the animal or towards the opening of the cage. The entire process was recorded using a video camera and the recordings were then analyzed and scored by a blinded experimenter. During each trial, the following responses were scored (1) ability of the animal to reach the front, left, or right side of the box, scored under the term “reach act”; (2) hand of choice for the reach, either left or right, scored under the term “hand used”; (3) the outcome of the reach, either success or failure, scored under result section.

Using the above parameters, additional variables were analyzed: 1) Motor problem: Reaching into the open side of the box but without retrieving the reward. 2) Initiation time: Latency from the screen being raised to the subject touching the box or reward. 3) Execution: Retrieving the reward from the box on the first reach of the trial (indicates competence on the task). 4) Correct: Eventually retrieving the reward from the box on the trial (>1 reach on the trial to retrieve the reward because unlimited reaches per trial were allowed). 5) Reach number: Number of times the animal made an attempt and touched the box. 6) Hand preference: Hand (left or right) subject used for the first reach of the trial. 7) Hand bias: Total number of left and right hand reaches on each trial. 8) Awkward reach: Reaching with the hand farthest away from the box opening (either the left or right side). 9) Perseverative response: Repeating a reach to the side of the box that was previously open but then closed. 10) Barrier reach: Reaching and touching the closed side of the test box. The results from the data analysis were plotted using Graph pad prism statistical software.

### Activity and Sleep Analysis

The diurnal and nocturnal behavior of the marmosets was monitored using the actiwatch mini (Camntech, UK) as previously described. The device is an accelerometer that measures the intensity of the test subject's omnidirectional movements in units or counts that are directly proportional to the animal's activity. The device (2 cm in diameter) was placed on a collar around the neck of the marmoset. The animals were acclimatized to the collar in short sessions of 15 min followed by gradual increments of 30 min, 1 hr, 3 hrs, 6 hrs and 12 hrs. Once acclimatized, the actiwatch mini was attached to the collar and the activity-rest data was recorded for a period of 24 hours on 3 separate days of each treatment. The actiwatches were placed on animals at 8:30 AM and the devices were preset to start recording activity data from 9:00 AM. At 11:30 AM either the vehicle or the drugs were orally administered to the animals. The following day, the actiwatch was removed after 9:00 AM and the data was transferred to a computer through an actiwatch reader using the actiwatch activity and sleep analysis-7 software (Camntech, UK). For sleep analysis, the period of sustained quiescence (marmosets sleep cycle) starting at 7:00 PM in the evening to early morning 6:30 AM (approximately 11½ hrs) was analyzed using the sleep analysis-7 software to quantify the sleep quality and wakeful periods. The duration of sleep time was corrected for individual variations in the animal's behavior to fall asleep at different time of the evening, thereby keeping the period of sleep time analyzed same for all the animals. The analyzed data was then exported to Excel® and plotted using the Graph pad prism statistical software. To determine the change in activity after the administration of drugs, actiwatch data in 10-minute bins were exported using the actiwatch activity and sleep analysis-7 software and plotted in Graph Pad Prism.

### Statistical Analysis

Statistical analysis was done with Graph Pad Prism statistical software. Significance in differences between 2 groups was performed by applying Student's t-test where appropriate. For comparison of multiple groups Two-Way ANOVA with Bonferroni post-hoc analysis was performed to identify the significant differences. A P-value of less than 0.05 was considered to be statistically significant.

### Docking Simulation for PD13R and SWR-3-73 Reveals Selectivity for D3R Versus D2R

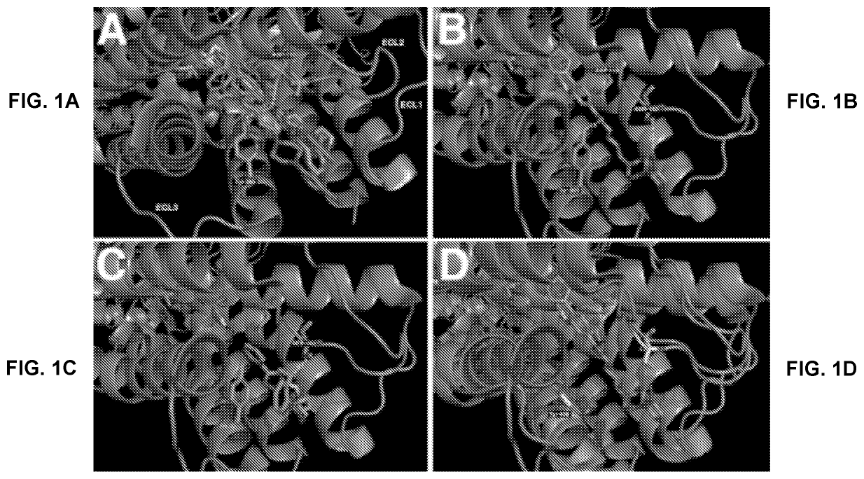

A potent D3R ligand, PD13R, was identified that is highly selective for the D3R over other dopamine receptors. Molecular modeling studies were performed to determine the binding region of PD13R and the D3R binding pocket. The Rhodium protein docking simulation program was used to implement a fully automated search over the agonist-bound D3R cryo-electron microscopy (cryo-EM) structure (PDB ID 7CMV) and predict the binding site. Rhodium's unique docking pose analysis is based on changes in free energy during ligand-protein interaction, which is utilized in affinity optimization in ligand design. The model focuses on cavity-filling and on matching hydrogen-bond donors and acceptors at the ligand-protein interface, rather on optimizing their counts. The docking site selection is based on the long-range potential rules and do not take in consideration analyst-chosen pockets. The optimal poses of PD13R revealed the structure of D3R bound to PD13R and localization of the amide group between TYR 365 and ASP 110 with polar contacts (FIG. 1A). The Asp-110 residue, located deep in the pocket, represents a crucial pharmacological site of the PD13R binding at the orthosteric binding site (OBS) of D3R. The lowest-energy pose with maximum cavity-filling is characterized by a short polar contact of the amide linker made with TYR 365 and by a longer contact made with SER 182 by the cyano group (FIG. 1B). The interactions of compound SWR-3-73 with D3R (magenta, FIG. 1C) displayed different energy profile with a short polar contact with the residue SER 182. Comparison with the off-target D2R cryo-EM structure (FIG. 1D) yields insights into selectivity of PD13R for D3R. In an overlay with the D2R structure (PDB 7JVR) the polar contacts to the key residues TYR 365 and SER 182 are lost. The TYR 365 became TYR 408 in D2R and reoriented completely away from the pocket leading to loss of the polar contact with the ligand. The overlay with the D2R structure revealed that the terminal methoxy group of compound SWR-3-73 is no longer in contact with the protein, leaving an un-paired hydrogen bond with no contact acceptor. The lack of selectivity for D2R caused by the impairment of hydrogen bonding either in the amide group of PD13R or in the terminal methoxy group of SWR-3-73 leads to a solvation penalty not present in the on-target D3R. Thus, selectivity is much higher towards D3R as compared to D2R because of the desolvation penalty for the two ligands PD13R and SWR-3-73 in D2R.

### Synthesis, Pharmacology and Mechanism of Action of PD13R

The synthesis of PD13R involved the conversion of 4-(thiophen-3-yl)benzoic acid to an amide by a reaction with 4-aminobutan-1-ol using the coupling agent 4-(4,6-dimethoxy-1,3,5-triazin-2-yl)-4-methyl-morpholinium chloride (DMT-MM). Carbon tetrabromide and triphenylphosphine were used to convert the DMT-MM to a primary bromide. The final reaction involved a nucleophilic displacement of the bromide with the arylpiperazine under basic conditions (K₂CO₃, CH₃CN).

PD13R was tested in the in vitro competitive D3R and D2R radioligand binding assays and rat liver microsome (RLM) stability assay. The D3R tolerated better the electron donating and electron withdrawing substituents on the phenyl ring of the arylpiperazine as indicated by the potent and high sub-nanomolar affinity for D3R with Ki=0.50 nM. The D3R over D2R data was as follows: D3R Ki=19.8 nM, D2R Ki>17,000 nM, D2R/hyperD3R>858.

Structure activity relationship (SAR) comparison between D3R versus D2R demonstrated that substitution in the 4-position of the phenyl ring of the arylpiperazine lead to substantial losses in D2 receptor binding irrespective of the electron character of the substituent and a 1486-fold high selectivity for D3R over D2R. This data is consistent with the docking results demonstrating that the phenylpiperazine region is critical to the binding, while the high degree of selectivity for D3R observed was driven by the optimal cavity filling influenced by the length of the spacer and the phenyl thiophene region.

To determine the selectivity of PD13R toward other DA receptors, an in vitro screen was performed for D1R, D4R, and D5R binding. The results showed a 1486-fold high selectivity for D3R over D2R, >20,000-fold selectivity for D3R over D1R or D5R and >1600-fold selectivity over D4R.

The ability of the compound PD13R to couple G-proteins in the forskolin-dependent adenylyl cyclase inhibition assay and the β-arrestin binding assay were then investigated. Quinpirole and haloperidol were used as a full agonist and antagonist, respectively. Compounds were tested for efficacy in these assay at a dose equal to 10 Å˜ their D3R Ki values in order to ensure >90% receptor occupancy and the results were compared with the impact of the full agonist. There was no activity in the β-arrestin binding assay and partial agonism in the forskolin-dependent adenylyl cyclase inhibition assay producing 19.4%±5.4 S.E.M., n=3) of the activity of the maximum response observed with quinpirole.

Table 1 presents the physicochemical and in vitro properties of PD13R. RLM and HLM are the half-lives for stability (minutes) using rat and human liver microsomes, respectively. Compounds were tested for activity at the D3 dopamine receptor at a dose 10× the Ki value of PD13R to ensure 90% of the sites were occupied. Compounds were evaluated for efficacy using adenylyl cyclase inhibition and β-arrestin binding assays (n=3). Values represent the percent of the maximum response where quinpirole was used as a full agonist reference compound and haloperidol was used as an antagonist.

TABLE 1

| RLM | HLM | D₁ | D₂ | D₃ | D₄ | D₅ | Camp | −Arrestin |
|---|---|---|---|---|---|---|---|---|
| T_{1/2} (min) | | Ki (nM) | | | | | 5 nM | |
| 60 | 60 | 10,000 | 743 | 0.5 | 10,000 | 10,000 | 19.4 ± 5.4 | −3.0 ± 1.4 |

### Development of Levodopa-Induced Dyskinesia in Parkinsonian Marmosets

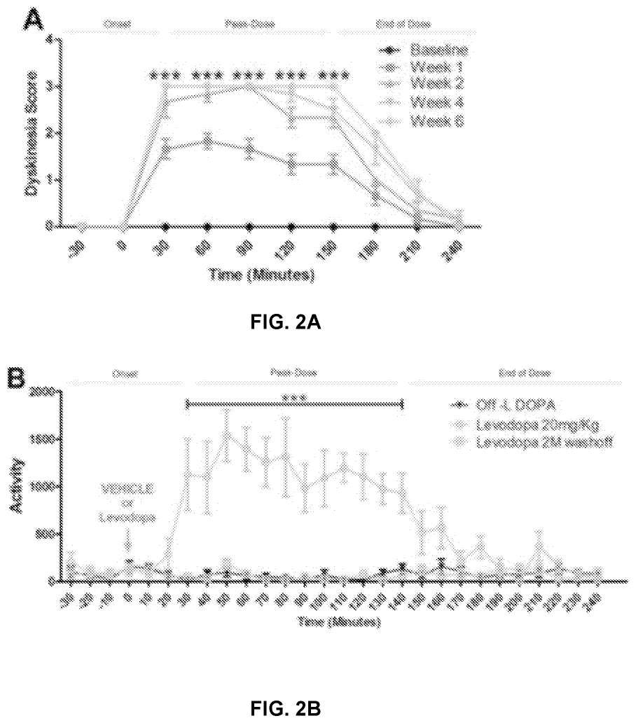

The motor and non-motor Parkinson's-like symptoms were previously characterized in the MPTP marmoset model including postural and action tremors, altered range of motion during reaching, bradykinesia and problem solving during the skilled action of retrieving a reward. These symptoms were improved with levodopa treatment. In this study, levodopa-induced dyskinesia was modelled, including debilitating side effects that PD patients develop after long-term levodopa pharmacotherapy. Previous studies have demonstrated that the marmosets readily develop levodopa-induced dyskinesia after extended period of levodopa treatment. To induce dyskinesia, the Parkinsonian marmosets were started on a low dose of levodopa/Carbidopa (1:1, 5 mg/kg. B. Wt.) followed by medium dose (10 & 15 mg/kg. B. Wt.). Once the animals acclimatized to the drugs, they were switched to high dose (20 mg/kg. B. Wt.) and maintained for 6 weeks. To measure dyskinesia, Dyskinesia disability rating scale was used that is scored for different segments of the animal's body including face, trunk, arms and legs (see prior section) and a wearable device, the actiwatch-mini to monitor the animals' activities while they are dyskinetic. During the low and medium dose of levodopa therapy, the Parkinsonian marmosets displayed a generalized increase in motility, absence of action tremors, and increase in vocalization with no apparent symptoms of dyskinesias. In the first week of high dose levodopa treatment, however, the Parkinsonian marmosets started displaying mild dyskinesia (FIGS. 2A and 2B) that manifested by transient abnormal movements, increased running and hopping in the cages. The majority of these abnormal involuntary movements were choreic with abnormal flicking of limbs and arms, which became visible approximately 30 minutes after dosing and lasted up to 180 minutes. In the following weeks however, the levodopa-induced dyskinesia gradually became more generalized with a steep increase in severity and total duration of symptoms (FIGS. 2A and 2B). Within 30 minutes of dosing, the marmosets started to manifest marked abnormal involuntary flowing movements of arms and limbs frequently tapping or touching cage walls or nest box. During the peak dose period, the Parkinsonian marmosets exhibited highest dyskinesia score and hyperactivity measured by actigraphy (FIGS. 2A and 2B). The dyskinesia consisted of sustained hyperkinesia with stereotypic repetitive waving movements of the forearms and limbs. Chorea and athetosis (sinuous writhing movements of the limbs) were increasingly common at this stage. Dystonic movements were exhibited as sustained extension of hind limbs and knees. During the 6^{th} week of high dose of levodopa therapy (20 mg/kg), dyskinesia lasted up to 4 hours after dosing with 3 well-delineated periods of dyskinesia, the onset period, the peak-dose period with maximum dyskinesia disability score and the end-of-dose period (FIGS. 2A and 2B). Treatment with the dopamine D3R ligand PD13R prevents the expression of levodopa-induced dyskinesia in the Parkinsonian marmosets.

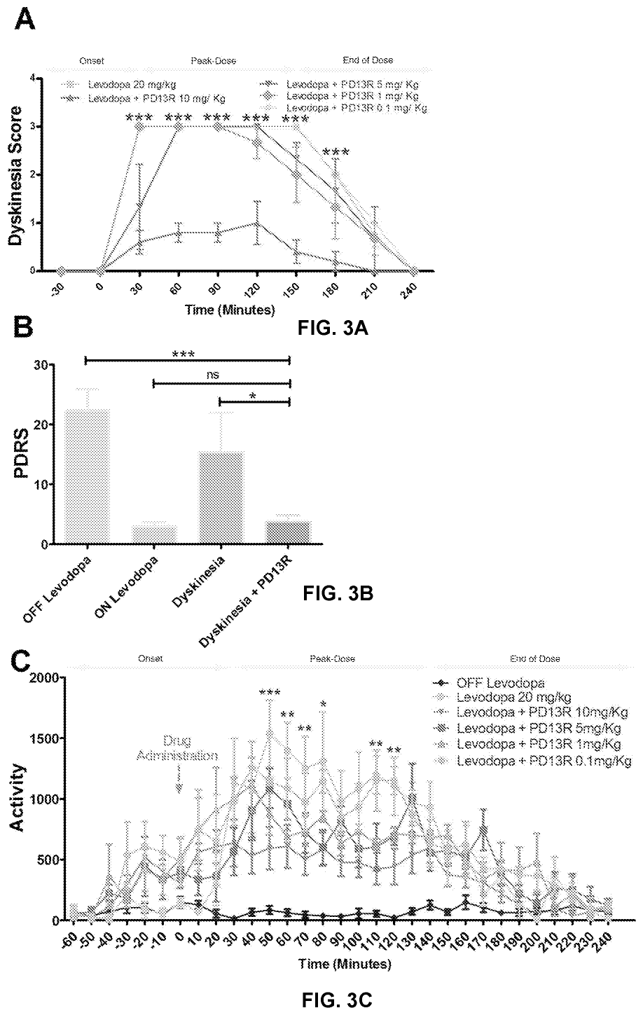

Marmosets were treated with dyskinesia-inducing high dose of levodopa (20 mg/kg) in combination with either vehicle or increasing doses of PD13R. Animal treated with levodopa combined with vehicle reached the peak-dose period of dyskinesias in 30 min after dosing with highest dyskinesia disability score of 3 and lasted for 2 hours. The marmoset model showed that the peak-dose dyskinesia and the ON response coincide following Levodopa treatment. This outcome of levodopa treatment is consistent with the outcome observed in patients with levodopa-induced dyskinesias. When the Parkinsonian marmosets were treated with levodopa (20 mg/kg) and PD13R at dose of 0.1 mg/kg no anti-dyskinetic effects were observed. While treatment with levodopa+PD13R at 1 mg/kg dose reduced the duration of the peak-dose period to 1.5 hours with a progressive decrease during the end-of-dose period in dyskinesia disability score (FIG. 3A). The combination of levodopa+PD13R at 5 mg/kg dose reduced the expression of levodopa-induced dyskinesia during the onset period, reaching peak-dose after 1 hour and reduced the peak levodopa effects to 1 hour before starting a progressive decrease (FIG. 3A). When used at a dose of 10 mg/kg in combination with levodopa, PDR13 drastically reduced the expression of dyskinesia by approximately 85% in the peak-dose period of levodopa-induced dyskinesia (FIG. 3A).

PD13R Improved the PDRS and Normalized Daily Activity of the Parkinsonian Marmosets with Levodopa-Induced Dyskinesia.

The anti-dyskinetic effects of PD13R that could lead to the improvement of the Parkinson's disease-like symptoms were investigated. The PDRS were first used to evaluate the Parkinsonian syndrome during levodopa-induced dyskinesia. The Parkinsonian marmosets displayed resting tremors, bradykinesia, hypokinesia and apathy with an average PDRS of 25 (FIG. 3B). High doses of Levodopa induced dyskinesias and worsened the Parkinsonian symptoms while physiological doses improved the PDRS (FIG. 3B). Importantly, PD13R treatment eliminated the levodopa-induced dyskinesia and significantly improved the PDRS (FIG. 3B). To further confirm the anti-Parkinsonian effects of PD13R, we used the actiwatch-mini and measured the animals' activities during dyskinesia. The Parkinsonian marmosets were treated with increasing doses of PD13R (0.1 to 10 mg/kg) in combination of dyskinesias-inducing high dose of levodopa (20 mg/kg). The actograms revealed a dose-dependent and significant reduction in the hyperactivity during the peak-dose period of levodopa-induced dyskinesia (FIG. 3C). PD13R improved the hyperactivity at 10 mg/kg and kept the motor activity within the ON-period with optimum anti-Parkinsonian and anti-dyskinetic beneficial effects enabling the animal to move around freely with motor complications.

### Comparative Anti-Dyskinetic Effects of PD13R, SWR373, and Amantadine

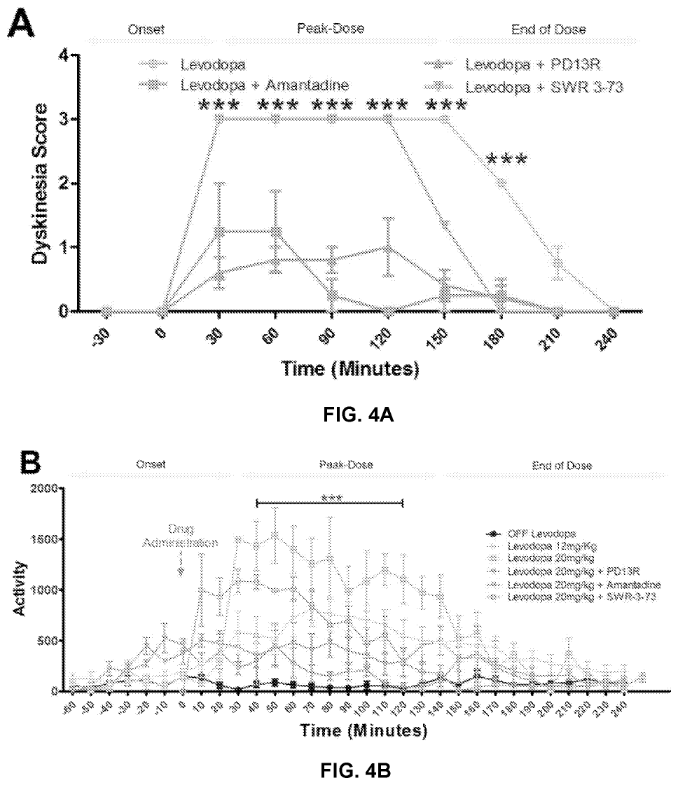

Amantadine is currently the main drug used in clinical practice under the name “GOCOVRI” or “ADS-5102” for treating levodopa-induced dyskinesias. The anti-dyskinetic beneficial effects of PD13R were compared to amantadine as a positive control and to a second D3R receptor partial agonist SWR-3-73. Parkinsonian marmosets were treated with dyskinesia-inducing high dose of levodopa (20 mg/kg) in combination with either amantadine (10 mg/kg), SWR373 (10 mg/kg) or with PD13R (10 mg/kg). The data showed that both amantadine and PD13R significantly inhibited the expression of dyskinesias throughout the onset, peak-dose and end-of-dose periods of levodopa treatment (FIG. 4A). While compound SWR-3-73 reduced the duration of the peak-dose period to 1.5 hours with an early and progressive decrease of the end-of-dose period of dyskinesia (FIG. 4A).

The anti-dyskinetic effects of PD13R, SWR-3-73 and amantadine were all compared at 10 mg/kg on the peak-dose levodopa-induced dykinesias using the actiwatch-mini (FIG. 4B). Both PD13R and amantadine inhibited the increased hyperactivity exhibited by the animals during the peak-dose period of levodopa-induced dyskinesias, while SWR-3-73 treated marmosets exhibited dyskinesia-associated hyperactivity during this period (FIG. 4B).

### Amantadine Causes Sleep Disturbances in Dyskinetic Parkinsonian Marmosets Under Levodopa Treatment

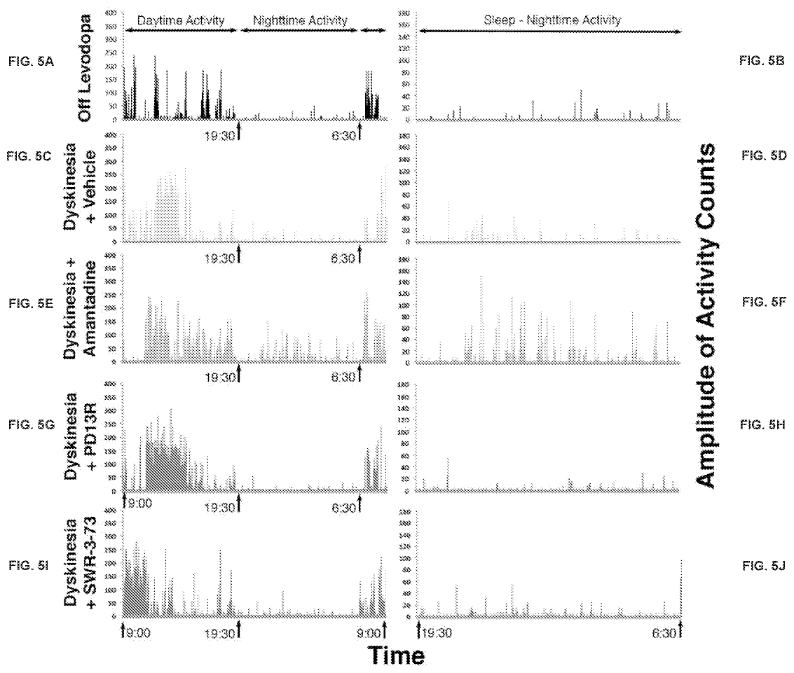

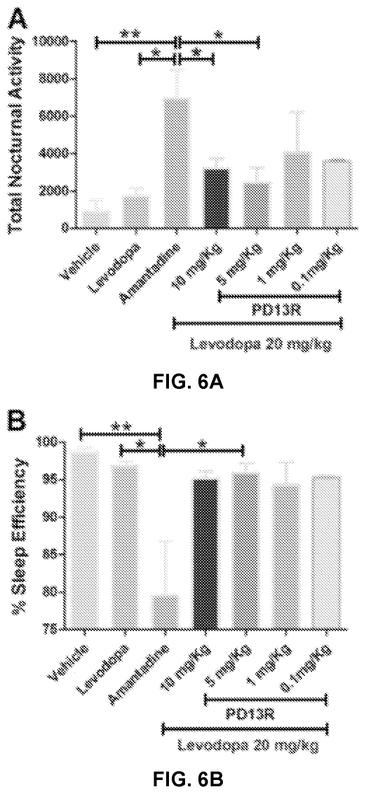

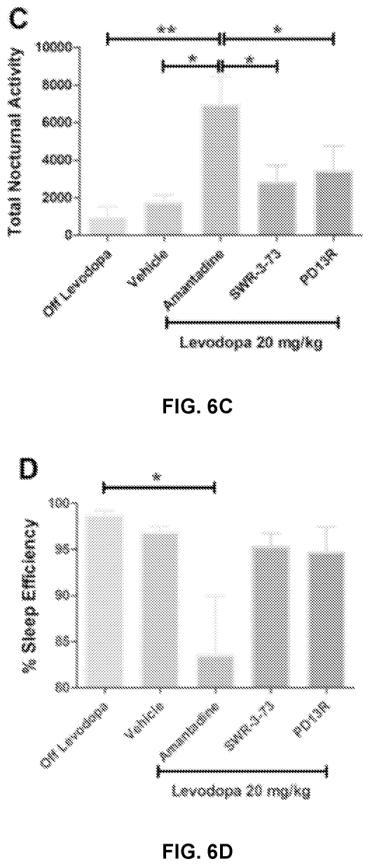

The actiwatch enable us to non-invasively and empirically analyze the activity and the quality of sleep during nighttime in dyskinetic marmosets treated with levodopa in combination with increasing doses of PD13R (0.1 to 10 mg/kg), with SWR-3-73, or with amantadine. Quantitative analysis of the actograms demonstrated significant increase in nocturnal activity, in time spent moving and wakefulness at nighttime in dyskinetic animals treated with amantadine compared to vehicle, to SWR373- or to PD13R-treated animals (FIGS. 5A-5J). We then investigated the soundness of sleep by analyzing the sleep efficiency and actual awake time during the assumed sleep period. The results showed that sleep efficiency significantly decreased in dyskinetic Parkinsonian marmoset treated with amantadine compared to vehicle-treated group and to dyskinetic animals treated with SWR-3-73 or with PD13R (FIGS. 6A-6C). Furthermore, the actual awake time was significantly increased in dyskinetic animals treated with amantadine compared to PD13R-treated animals suggesting the marmosets woke up more often at night under amantadine treatment. Together these results suggested that dyskinetic Parkinsonian marmosets treated with amantadine experienced abnormal irregular sleep and sleep disturbances while both D3R ligands, SWR373 and PD13R did not affect sleep.

In summary, numerous benefits have been described which result from employing the concepts described herein. The foregoing description of the one or more forms has been presented for purposes of illustration and description. Modifications or variations are possible in light of the above disclosure.

What is claimed is:

1. A method of treating dyskinesia in a subject in need thereof, the method comprising:
administering to the subject an effective amount of a dopamine receptor 3 ligand containing an arylpiperazine pharmacophore or a pharmaceutically acceptable salt, derivative, hydrate or solvate thereof.

2. The method of claim 1, wherein the dyskinesia is levodopa-induced dyskinesia.

3. The method of claim 1, wherein the dopamine receptor 3 ligand further contains a phenyl thiophene moiety.

4. The method of claim 1, wherein the dopamine receptor 3 ligand is a compound represented by Formula I:

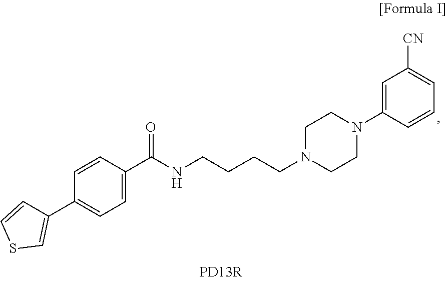 [SMILES: N#Cc1cccc(N2CCN(CCCCNC(=O)c3ccc(-c4ccsc4)cc3)CC2)c1]

5. The method of claim 4, wherein the dopamine receptor 3 ligand is administered orally.

6. The method of claim 4, wherein the dopamine receptor 3 ligand is administered as a thiophenyl benzoate salt of the compound represented by Formula I.

7. A method of treating levodopa-induced dyskinesia in a subject in need thereof, the method comprising:
administering to the subject an effective amount of a dopamine receptor 3 ligand containing N-(4-(4-phenyl piperazin-1-yl)butyl)-4-(thiophen-3-yl)benzamide or a pharmaceutically acceptable salt, derivative, hydrate or solvate thereof.

8. The method of claim 7, wherein the dopamine receptor 3 ligand containing N-(4-(4-phenyl piperazin-1-yl)butyl)-4-(thiophen-3-yl)benzamide or a pharmaceutically acceptable salt, derivative, hydrate or solvate thereof is administered orally.

9. The method of claim 7, wherein the dopamine receptor 3 ligand containing N-(4-(4-phenyl piperazin-1-yl)butyl)-4-(thiophen-3-yl)benzamide or a pharmaceutically acceptable salt, derivative, hydrate or solvate thereof is administered as a thiophenyl benzoate salt.

10. The method of claim 7, wherein the dopamine receptor 3 ligand is administered at a dosage ranging from about 1 mg/kg to about 20 mg/kg.
